(19) United States
(12) Patent Application Publication
(10) Pub. No.: US 2015/0283320 A1
(43) Pub. Date: Oct. 8, 2015

(54) MEDICAL TREATMENT METHOD, MEDICAL TREATMENT APPARATUS, AND MEDICAL TREATMENT SYSTEM FOR BLOODSTREAM DISORDER

(71) Applicant: SEIKO EPSON CORPORATION, Tokyo (JP)

(72) Inventors: Hiroshi KUNIKATA, Sendai-shi (JP); Yuji TANAKA, Yokohama-shi (JP); Toru NAKAZAWA, Sendai-shi (JP); Atsuhiro NAKAGAWA, Sendai-shi (JP); Teiji TOMINAGA, Sendai-shi (JP); Hideki KOJIMA, Matsumoto-shi (JP)

(21) Appl. No.: 14/677,918

(22) Filed: Apr. 2, 2015

(30) Foreign Application Priority Data
Apr. 3, 2014 (JP) 2014-076593

(51) Int. Cl. A61M 5/14

(52) U.S. Cl. CPC A61M 5/14

(57) ABSTRACT

Occlusion of a blood vessel is improved by a medical treatment system including a ejection device that includes an insertion section insertable into a tissue of an animal and is capable of ejecting first liquid as a pulsating flow from a distal end of the insertion section and a control apparatus configured to bring the distal end of the insertion section close to a blood vessel in the tissue, which is a medical treatment target, and eject the first liquid as the pulsating flow from the distal end of the insertion section in a state in which second liquid is interposed in a region covering the blood vessel.

This application claims the benefit of Japanese Patent Application No. 2014-076593 filed on Apr. 3, 2014. The content of the aforementioned application is incorporated herein by reference in its entirety.

### BACKGROUND

1. Technical Field

The present invention relates to a medical treatment technique for a bloodstream disorder.

2. Related Art

There is a bloodstream disorder caused by the presence of an object that obstructs a flow of blood in a blood vessel such as a blood clot or an embolus. As a medical treatment method in the past, in order to remove a blood clot and an embolus and recover a bloodstream, there have been proposed, for example, a method of administering a drug to melt the blood clot or the embolus, a method of inserting a catheter into the blood vessel to crush or suck the blood clot or the embolus, and a method of spraying an intermittent jet stream on the outside of the blood vessel (JP-T-2003-500098 (Patent Literature 1).

When the intermittent jet stream is sprayed on the outside of the blood vessel, clotted blood that occurs in the blood vessel is pushed down in a downstream direction by the jet flow and occlusion is eliminated. In such a method, it is unnecessary to insert the catheter or the like into the blood vessel. By simply massaging the blood vessel (mainly a vein), it is possible to treat the occlusion of the vein. Therefore, this method is considered to be noninvasive for the blood vessel.

However, the methods explained above respectively have problems explained below. When the drug for melting is administered, the administration is limited because of the influence on a patient. In the method of using the catheter, facilities capable of carrying out the method are limited because a sophisticated technique is necessary. In particular, it is difficult to use the drug administration and the catheter for an extremely thin blood vessel. For example, adaptation to, for example, capillaries on a retina of an eyeball necessary for medical treatment of retinal vein occlusion is difficult.

On the other hand, in the method of ejecting high-pressure liquid in a pulse-like manner, a massage effect is high through a cyclical shock. However, it is likely that the blood vessel or tissues near the blood vessel are damaged (sometimes injured) by the shock. Besides, in a medical treatment method and a medical treatment apparatus in the past, there has been a demand for simplification of the medical treatment method and the medical treatment apparatus, a reduction in the size of the apparatus, a reduction in costs, resource saving, simplification of manufacturing, improvement of convenience of use, and the like.

### SUMMARY

An advantage of some aspects of the invention is to solve at least a part of the problems described above, and the invention can be implemented as the following aspects.

(1) A first aspect of the invention provides a medical treatment method for a bloodstream disorder. The medical treatment method includes: preparing a ejection device that includes an insertion section insertable into a tissue of an animal and is capable of ejecting first liquid as a pulsating flow from a distal end of the insertion section; and bringing the distal end of the insertion section close to a blood vessel in the tissue, which is a medical treatment target, and ejecting the first liquid as the pulsating flow from the distal end of the insertion section in a state in which second liquid is interposed in a region covering the blood vessel.

With the medical treatment method, a shock due to the ejected first liquid is reduced by the second liquid present in the region covering the blood vessel and does not excessively damage the blood vessel.

(2) In the medical treatment method, the first liquid and the second liquid may be the same liquid. If the first liquid and the second liquid are the same liquid, it is easy to prepare the liquid. After the ejecting, the ejected first liquid is mixed with the second liquid and functions as the second liquid. The first liquid can be used to supplement the second liquid. Note that the first liquid and the second liquid may be different liquids. In this case, roles of the two liquids are varied and respective effects of the liquids can be educed. For example, methods of use such as concentration adjustment of a drug (when drug concentrations of the first and second liquids are different) and visual recognition of an ejection state of the first liquid (when colors and degrees of clearness of the first and second liquids are different) are conceivable.

(3) In the medical treatment method, prior to the ejection of the first liquid, the second liquid may be filled in the region covering the blood vessel. It is also likely that liquid such as lymph fluid covers the blood vessel and it is unnecessary to fill the second liquid in advance. However, when the blood vessel is not covered by the liquid or when the liquid needs to be replaced even if the blood vessel is covered by the liquid, prior to the ejection of the first liquid, the second liquid only has to be filled in the region covering the blood vessel.

(4) In the medical treatment method, prior to the filling of the second liquid, at least a part of the tissue present in the region covering the blood vessel may be excised to form a space in which the second liquid is filled. Since an eyeball or the like is filled with a vitreous body, if it is attempted to eject the first liquid on a retinal vein, it is necessary to excise the vitreous body covering the retinal vein and prepare a space in which the second liquid is filled. In such a case, at least a part of the tissue only has to be excised using an excising tool such as a vitreous body cutter to form the space. Note that excising means is not limited to the vitreous body cutter. Suitable excising means only has to be used according to characteristics of a tissue to be excised. Various excising means such as a laser knife, an electric knife, and an excising tool by a pulse jet can be used.

(5) In the medical treatment method, when the ejection of the first liquid is performed, an increase in pressure in the region where the second liquid is interposed may be reduced. If the first liquid continues to be ejected in a space having a limited volume, it is likely that the pressure in the region rises. In such a case, if the pressure is within an allowable range, the pressure rise may be left as it is or may be reduced. If the pressure rise is reduced, it is possible to avoid a risk of damage due to excessively high pressure on the tissue.

(6) The reduction in the pressure rise can be performed by discharge of the second liquid from the region. This is because, if the second liquid is discharged, the pressure falls.

(7) Alternatively, the reduction in the pressure rise can also be performed by increasing or reducing, according to the pressure in the region, a volume of a pressure reduction chamber provided in the region. The reduction in the pressure rise may be performed by other methods.

(8) Pressure in the region may be monitored to carry out at least one of adjustment of the pressure based on fluctuation in the pressure during medical treatment, a stop of the ejection of the first liquid, and provision of the pressure fluctuation during the medical treatment to a surgeon. It is possible to cope with the pressure rise with any one of the methods.

(9) In the medical treatment method, the tissue of the animal may be an eyeball, the blood vessel may be a retinal vein of the eyeball, and the bloodstream disorder may be retinal vein occlusion. In the medical treatment method, a shock of the ejection of the first liquid is reduced by the second liquid. Therefore, when the medical treatment method is applied to medical treatment of occlusion of a fragile blood vessel such as the retinal vein, an effect of the medical treatment method is conspicuous.

(10) A second aspect of the invention provides a medical treatment apparatus used for the medical treatment method explained above. The medical treatment apparatus includes a ejection device that includes an insertion section insertable into a tissue of an animal and is capable of ejecting first liquid as a pulsating flow from a distal end of the insertion section. With the medical treatment apparatus, since the medical treatment apparatus includes the insertion section insertable into the tissue of the animal, by inserting the insertion section close to a blood vessel, which is a medical treatment target, and ejecting the first liquid into the second liquid from the distal end of the insertion section as the pulsating flow, it is possible to use the medical treatment apparatus for medical treatment for reducing occlusion of the blood vessel.

(11) A third aspect of the invention provides a medical treatment system for a bloodstream disorder. The medical treatment system includes: a ejection device that includes an insertion section insertable into a tissue of an animal and is capable of ejecting first liquid as a pulsating flow from a distal end of the insertion section; and a control apparatus configured to bring the distal end of the insertion section close to a blood vessel in the tissue, which is a medical treatment target, and eject the first liquid as the pulsating flow from the distal end of the insertion section in a state in which second liquid is interposed in a region covering the blood vessel.

The medical treatment system can easily bring the insertion section, from which the first liquid is ejected, close to the medical treatment target blood vessel. When the first liquid is ejected as the pulsating flow in that state, a shock of the ejection of the first liquid is reduced by the second liquid present in the region covering the blood vessel. Therefore, it is possible to use the medical treatment system for medical treatment that does not excessively damage the blood vessel.

(12) The medical treatment system may further include an excising apparatus configured to excise the tissue present in the region covering the blood vessel and form a region where the second liquid is stored. With the medical treatment system, it is possible to actively form the region where the second liquid is stored.

(13) Alternatively, the medical treatment system may further include a liquid supplying apparatus configured to fill the second liquid in the formed region. With the medical treatment system, it is possible to easily interpose the second liquid in the region covering the blood vessel.

(14) The medical treatment system may further include a pressure detecting apparatus configured to detect pressure of the second liquid interposed in the region covering the blood vessel. If the pressure is detected, it is possible to take appropriate measures against pressure fluctuation, in particular, a pressure rise.

(15) The control apparatus may control an ejection amount of the first liquid from the ejection device using the detected pressure. Consequently, it is possible to reduce fluctuation of the pressure according to the ejection amount of the first liquid.

Not all of the plurality of components included in the aspects of the invention explained above are essential. In order to solve a part or all of the problems explained above or attain a part of all of the effects described in this specification, concerning apart of the plurality of components, it is possible to appropriately perform a change, deletion, and replacement of the components with other components and deletion of a part of limitation contents. In order to solve a part or all of the problems explained above or in order to attain a part or all of the effects described in this specification, it is possible to combine a part or all of the technical features included in one aspect of the invention explained above with a part or all of the technical features included in the other aspects of the invention to form an independent one aspect of the invention.

The invention can also be implemented as various forms other than the medical treatment apparatus and the medical treatment system. The invention can be implemented informs of, for example, a manufacturing method for the medical treatment apparatus and a control method for the medical treatment apparatus, a computer program for implementing the control method, and a non-transitory recording medium having the computer program recorded therein.

### BRIEF DESCRIPTION OF THE DRAWINGS

The invention will be described with reference to the accompanying drawings, wherein like numbers reference like elements.

FIG. 1 is a schematic configuration diagram showing a medical treatment system for a bloodstream disorder according to a first embodiment.

FIG. 2 is an enlarged sectional view of a part of the internal configuration of a hand piece.

FIG. 3 is an explanatory diagram showing an example of a waveform of a drive voltage applied to a piezoelectric element.

FIG. 4 is an explanatory diagram showing a correspondence relation between the waveform of the drive voltage and deformation of a diaphragm.

FIG. 5 is a process chart of a medical treatment method for retinal vein occlusion.

FIG. 6 is an explanatory diagram showing a first process in a medical treatment method for retinal vein occlusion.

FIG. 7 is an explanatory diagram showing a second process in the medical treatment method for retinal vein occlusion.

FIG. 8 is an explanatory diagram showing a third process in the medical treatment method for retinal vein occlusion.

FIG. 9 is an explanatory diagram showing a fourth process in the medical treatment method for retinal vein occlusion.

FIG. 10 is a schematic configuration diagram showing a medical treatment system for a bloodstream disorder according to a second embodiment.

FIG. 11 is an explanatory diagram for explaining a state of medical treatment in the second embodiment.

FIG. 12 is a flowchart for explaining the operation of a control apparatus in the second embodiment.

FIG. 13 is an explanatory diagram showing a main part in a modification 1.

FIG. 14 is an explanatory diagram showing a modification 2.

FIG. 15 is an explanatory diagram showing a modification 3.

### DESCRIPTION OF EXEMPLARY EMBODIMENTS

### A. First Embodiment

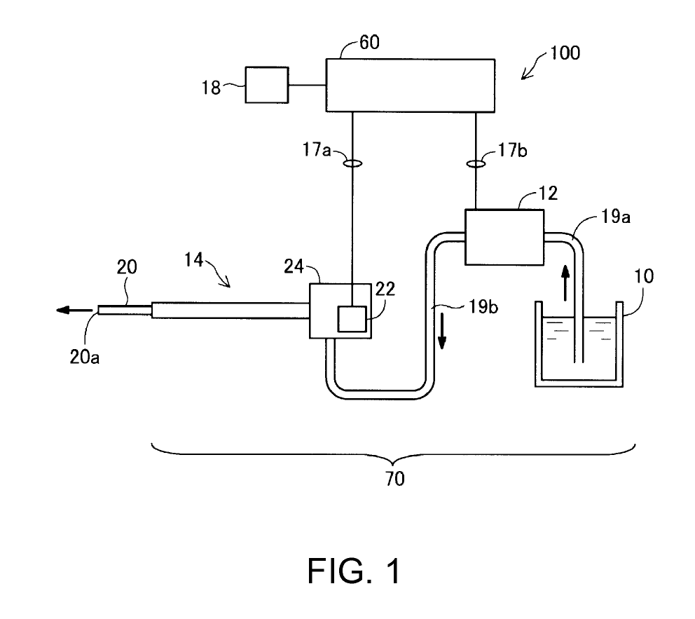

Embodiments of the invention are explained below with reference to the drawings. FIG. 1 is an explanatory diagram showing the configuration of a medical treatment system 100 for retinal vein occlusion according to a first embodiment of the invention. The medical treatment system 100 includes a liquid ejection device 70 that ejects liquid on an occluded region and a control apparatus 60 that controls the ejection. The medical treatment system 100 in this embodiment is a medical apparatus used in a medical institution. The medical treatment system 100 has a function of performing medical treatment by ejecting liquid on a biological tissue, which is an affected part of a patient. In particular, the medical treatment system 100 in the first embodiment is configured as a medical treatment system for retinal vein occlusion.

The liquid ejection device 70 includes a liquid container 10, a liquid supply mechanism 12, and a hand piece 14. The liquid container 10 and the liquid supply mechanism 12 are connected by a connection tube 19a. The liquid supply mechanism 12 and the hand piece 14 are connected by a connection tube 19b. In this embodiment, the connection tubes 19a and 19b are formed of resin.

In this embodiment, the liquid container 10 stores a diluted solution of an oxiglutatione solution as liquid supplied to the hand piece 14. As the liquid stored in the liquid container 10, liquid corresponding to a purpose of a surgical operation is used. In the case of a surgical operation of an eyeball, in general, the diluted solution of the oxiglutatione solution is used. However, depending on a surgical part, other liquids that are harmless even if ejected on a biological tissue such as saline, a Ringer's solution, pure water, and a drug solution only have to be used.

The liquid supply mechanism 12 supplies the liquid stored in the liquid container 10 to the hand piece 14 via the connection tubes 19a and 19b. In this embodiment, a pump is used as the liquid supply mechanism 12. In this embodiment, when receiving an instruction from the control apparatus 60, the liquid supply mechanism 12 supplies the liquid to the hand piece 14 at a fixed flow rate.

The hand piece 14 is an instrument held and operated by a surgeon. The hand piece 14 includes a liquid ejecting needle 20, a pulsation generating unit 22, and a housing 24. The liquid is supplied to the pulsation generating unit 22 via the connection tube 19b. When a drive voltage is applied to the pulsation generating unit 22 from the control apparatus via a voltage application cable 17a, the pulsation generating unit 22 applies pulsation to the supplied liquid. The liquid applied with the pulsation is ejected from an opening 20a at the distal end of the liquid ejecting needle 20 in an axial direction of the needle. Note that “pulsation” means that the liquid flows while involving pressure fluctuation. “Pulsation” includes a variety of ejecting forms such as a form in which ejection is completely interrupted between ejection and ejection and a form in which a low-pressure flow is present even between ejection and ejection. The frequency and the duty of the pulsation may be fixed or may change during ejection. The control apparatus 60 may vary these forms.

The control apparatus 60 includes a not-shown microcomputer incorporating or externally attached with a memory, an interface circuit with the outside, and various drive circuits. A footswitch 18 is connected to the control apparatus 60. When the footswitch 18 is operated by a surgeon, the control apparatus 60 detects the operation via the interface circuit and applies a drive signal from an incorporated drive circuit (not shown in the figure) to the pulsation generating unit 22 via the voltage application cable 17a. Further, the control apparatus 60 outputs, according to the output of the drive signal to the pulsation generating unit 22, a control signal to the liquid supplying mechanism 12 via a control cable 17b and controls the start and the stop of the liquid supply mechanism 12.

The footswitch 18 is a switch operated by the foot of the surgeon. When the surgeon turns on the footswitch 18, the control apparatus 60 instructs the liquid supply mechanism 12 to start the supply of the liquid and applies the drive voltage to the pulsation generating unit 22. The liquid applied with the pulsation is ejected from the opening 20a at the distal end of the liquid ejecting needle 20 provided in the hand piece 14.

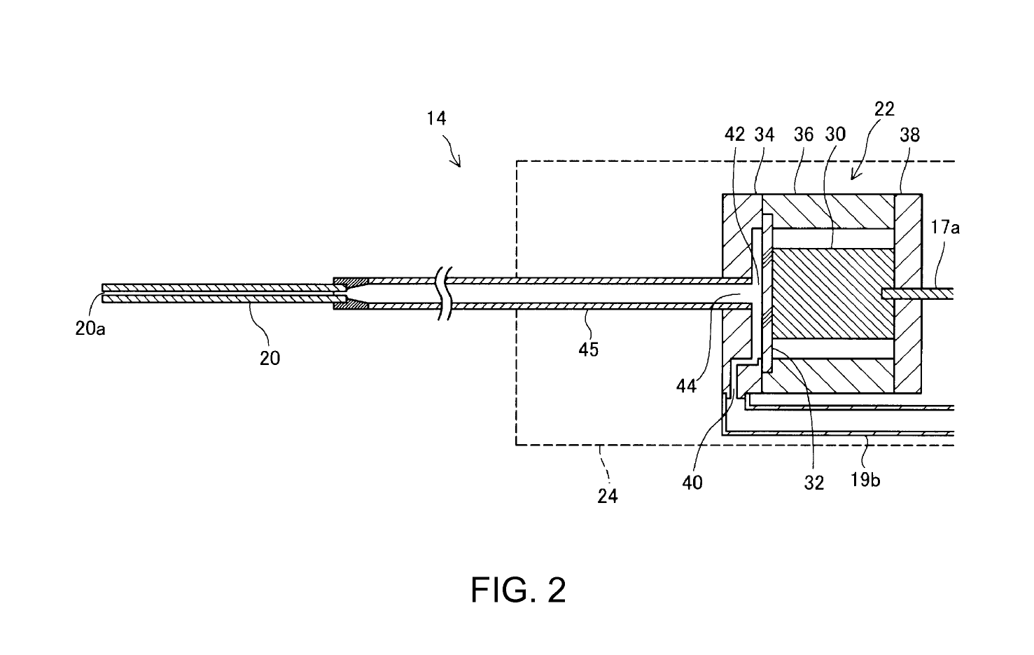

FIG. 2 is an enlarged sectional view of a part of the internal configuration of the hand piece 14. The pulsation generating unit 22 that applies pulsation to the liquid, which is supplied from the liquid supply mechanism 12, is provided on the inside of the housing 24 of the hand piece 14. The pulsation generating unit 22 includes a piezoelectric element 30, a diaphragm 32, a first case 34, a second case 36, and a third case 38.

On the inside of the pulsation generating unit 22, an inlet channel 40, a liquid chamber 42, and an outlet channel 44 are formed as a channel through which the liquid supplied from the liquid supply mechanism 12 passes. In this embodiment, the inlet channel 40 and the outlet channel 44 are formed in the first case 34. The liquid chamber 42 is formed between the first case 34 and the diaphragm 32. The connection tube 19b is connected to the inlet channel 40. The liquid ejecting needle 20 is connected to the outlet channel 44 via an outlet pipe 45.

The liquid ejecting needle 20 is a thin tube made of stainless steel. In this embodiment, a thin tube of a 25 gauge (an external diameter is 0.5 mm and an internal diameter is 0.32 mm). The length of the liquid ejecting needle 20 is about 20 mm. In FIG. 2, the liquid ejecting needle 20 is directly connected from the outlet channel 44. The length of the liquid ejecting needle 20 may be about several millimeters to several tens millimeters. The outlet channel 44 and the liquid ejecting needle 20 may be connected by a metal pipe having a large orifice size or hard plastics. The thickness of the liquid ejecting needle 20 only has to be selected according to a clinical case between a degree of a rather-thick 20 gauge and a degree of a rather-thin 30 gauge. It goes without saying that optimum thickness only has to be selected taking account of ejection pressure from the liquid ejection device 70 as well.

The diaphragm 32 of the pulsation generating unit 22 is a disk-like metal thin plate. The outer circumferential portion of the diaphragm 32 is held and fixed between the first case 34 and the second case 36.

The piezoelectric element 30 is an actuator that operates with the drive voltage applied from the control apparatus 60. The piezoelectric element 30 changes the pressure of the liquid in the liquid chamber 42 by changing the volume of the liquid chamber 42 formed between the diaphragm 32 and the first case 34. In this embodiment, the piezoelectric element 30 is a stacked piezoelectric element. One end of the piezoelectric element 30 is fixed to the diaphragm 32 and the other end of the piezoelectric element 30 is fixed to the third case 38.

When the drive voltage applied to the piezoelectric element 30 increases, the piezoelectric element 30 extends and the diaphragm 32 is pushed by the piezoelectric element 30 and bends to the liquid chamber 42 side. When the diaphragm 32 bends to the liquid chamber 42 side, the volume of the liquid chamber 42 decreases. The liquid in the liquid camber 42 is pushed out from the liquid chamber 42. In this embodiment, the inner diameter of the outlet channel 44 is larger than the inner diameter of the inlet channel 40. That is, since the inertance of the outlet chamber 44 is smaller than the inertance of the inlet chamber 40, the liquid in the liquid chamber 42 is pushed out from the liquid chamber 42 through the outlet channel 44.

On the other hand, when the drive voltage applied to the piezoelectric element 30 decreases, the piezoelectric element 30 contracts, the volume of the liquid chamber 42 increases, and the liquid is supplied from the inlet channel 40 into the liquid chamber 42.

The drive voltage applied to the piezoelectric element 30 repeats ON (maximum voltage) and OFF (0 V) at a specific frequency (e.g., 50 Hz). Therefore, the increase and the decrease of the volume of the liquid chamber 42 are repeated. Pulsation is applied to the liquid. The liquid pushed out from the liquid chamber 42 is ejected from a nozzle 20a (the opening 20a) at the distal end of the liquid ejecting needle 20.

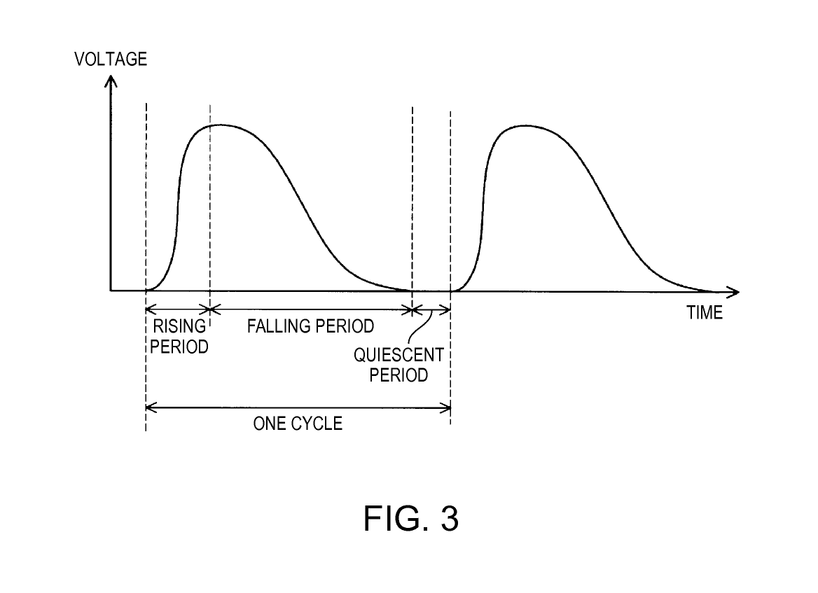

FIG. 3 is an explanatory diagram showing an example of a waveform of the drive voltage applied to the piezoelectric element 30. In FIG. 3, the abscissa indicates time and the ordinate indicates a drive voltage. One cycle of the waveform of the drive voltage includes a rising period in which the voltage increases, a falling period in which the voltage decreases, and a quiescent period in which the voltage is not applied.

In this embodiment, a waveform in the rising period of the drive voltage is a waveform for a half cycle of an SIN waveform offset in a positive voltage direction and phase-shifted by −90 degrees. A waveform in the falling period of the drive voltage is a waveform for a half cycle of an SIN waveform offset in the positive voltage direction and phase-shifted by +90 degrees. A cycle of the SIN waveform in the falling period is larger than a cycle of the SIN waveform of the rising period.

In this embodiment, when the magnitude of the drive voltage is changed by a condition switching unit 26, a maximum of the waveform shown in FIG. 3 is changed. When the frequency of the drive voltage is changed by the condition switching unit 26, the waveforms in the rising period and the falling period are not changed and the length of the quiescent period is changed.

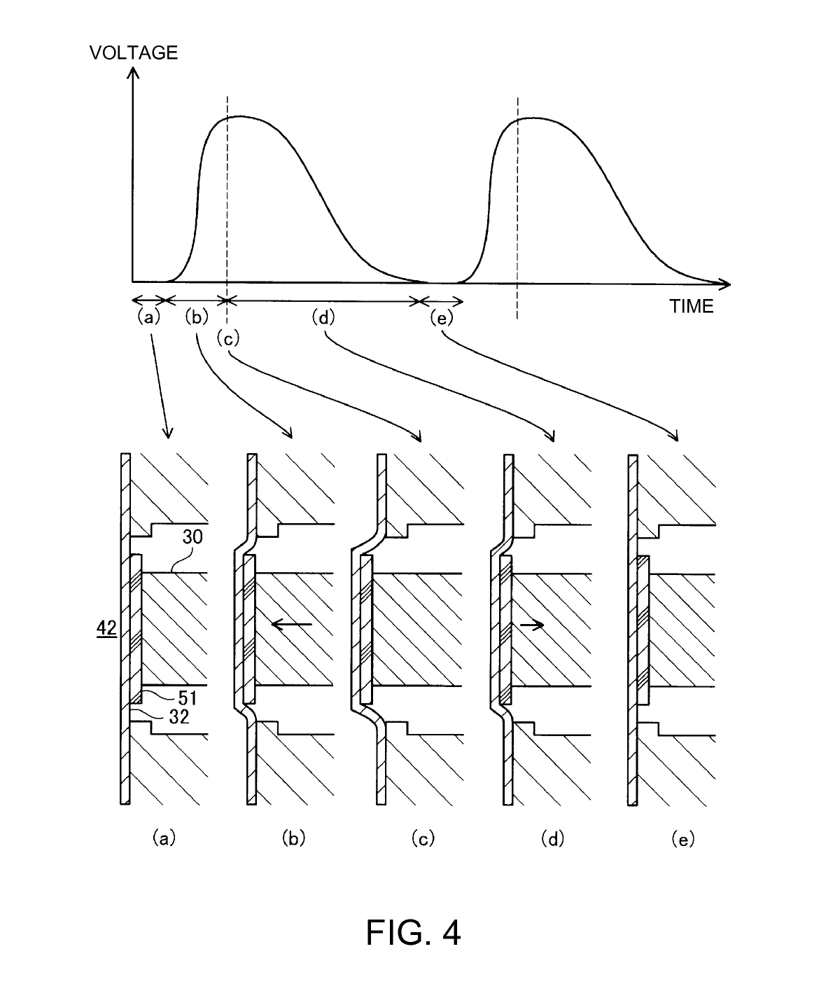

FIG. 4 is an explanatory diagram showing a correspondence relation between the waveform of the drive voltage and a state of deformation of the diaphragm 32. Note that, in FIG. 4, a reinforcing member 51 is provided between the piezoelectric element 30 and the diaphragm 32. In a quiescent period (a), since the drive voltage is not applied, the piezoelectric element 30 does not extend and the diaphragm 32 does not bend. In a rising period (b), since the drive voltage increases, the piezoelectric element 30 extends, the diaphragm 32 bends to the liquid chamber 42 side, and the volume of the liquid chamber 42 decreases.

At timing indicated by (c), since the drive voltage is the maximum, the length of the piezoelectric element 30 is also the maximum and the volume of the liquid chamber 42 is the minimum. In a falling period (d), since the drive voltage decreases, the piezoelectric element 30 starts to return to the original size and the volume of the liquid camber 42 starts to return to the original volume. In a quiescent period (e), since the drive voltage is not applied, the piezoelectric element 30 returns to the original size and the volume of the liquid chamber 42 returns to the original volume. The series of operations shown in (a) to (e) is repeated, whereby the liquid in the liquid chamber 42 is pushed out to the liquid ejecting needle 20.

### B. Medical Treatment of Retinal Vein Occlusion

A method of performing medical treatment of retinal vein occlusion using the medical treatment system 100 is explained. In the retinal vein occlusion, a part of a retinal vein distributed over an entire retina occludes, whereby a bloodstream is lost and reduced vision and a visual field defect are caused. The retinal vein occlusion is a disease widely found in animals (animals including human and mammals other than the human) having eyeballs. The occlusion of the retinal vein is mainly caused by a blood clot but is also caused by an embolus other than the blood clot. Note that the retinal vein occlusion is sometimes classified into a retinal vein ramification occlusion, retinal center vein occlusion, and the like. However, in this embodiment, the retinal vein occlusion is referred to as retinal vein occlusion irrespective of an occlusion position.

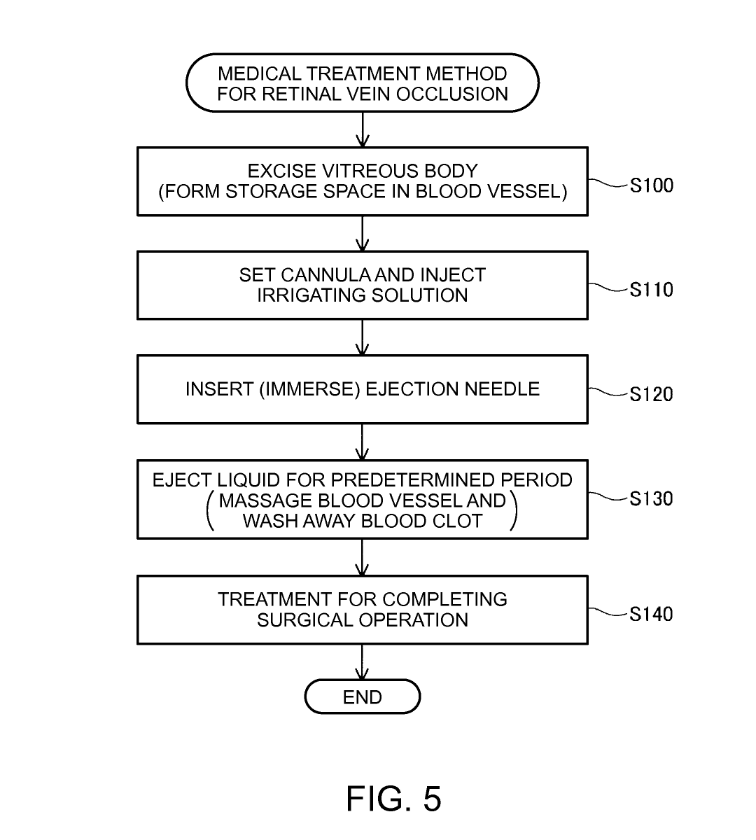

FIG. 5 is a process chart for explaining, as steps S100 to S140, the method of treating retinal vein occlusion using the medical treatment system 100. For convenience of explanation, the method is divided into five steps and explained. However, these steps are not always strictly divided. A part of the steps is sometimes simultaneously performed. It is also likely that the method is performed together with medical treatment of other diseases. It is also likely that, for example, excision of a vitreous body is performed as a part of medical treatment of other diseases. Further, although not shown in FIG. 5, in the medical treatment, for example, general anesthesia or regional anesthesia of a patient, cleaning and sterilization of a field of operation, and attachment of a monitor device are performed according to necessity. However, explanation thereof is omitted.

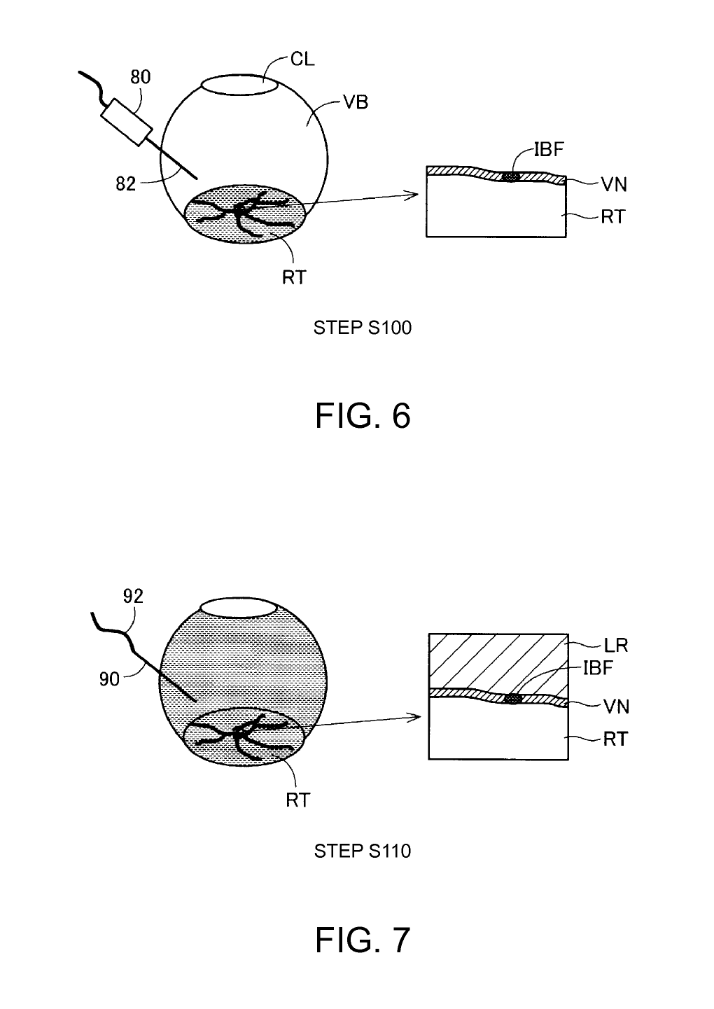

When the medical treatment is started, first, the surgeon performs treatment for excising the vitreous body (step S100). This state of the treatment is shown in FIG. 6. FIGS. 6 to 9 show the steps of the medical treatment. The left side of the figures shows an eyeball schematically and in perspective. The right side of the figure shows the vicinity of a retinal vein VN, which is a treatment target, as a sectional view. The eyeball includes a crystalline lens CL and a vitreous body VB. A retina RT is present on a so-called eyeground. A large number of the retinal veins VN are present in the retina RT. The vitreous body VB is a transparent jelly-like tissue, which fills a lumen of the eyeball, and is made of protein (collagen). A region clogged by a blood clot or the like (hereinafter referred to as bloodstream disorder region) IBF in the retinal vein VN is specified in advance by a funduscopy. The vitreous body VB present in an upper region of the bloodstream disorder region IBF is excised using a vitreous body cutter 80. The vitreous body cutter 80 is formed as a cutter section 82 having a sharp tip. The cutter section 82 includes a suction port for sucking and discharging the excised vitreous body VB.

The thickness of the cutter section 82 of the vitreous body cutter 80 is a 20 to 25 gauge. The cutter section 82 can easily enter the vitreous body VB. After the tip of the cutter section 82 is brought close to the vicinity of the bloodstream disorder region IBF, the vitreous body cutter 80 is actuated to excise the vitreous body VB. As the vitreous body cutter 80, there are vitreous body cutters of various types. In this embodiment, a vitreous body cutter of a type for outputting an ultrasonic wave from a distal end to emulsify the vitreous body VB and sucking the vitreous body VB from a suction port provided at the distal end is used. Besides, vitreous body cutters of all types such as a type for removing the vitreous body VB with rotation of a distal end and a type for excising the vitreous body VB with high-speed vibration of an excision blade provided at a distal end can be used. By excising the vitreous body VB in this way, a storage space LR for the liquid is formed in an upper part of the blood vessel VN where the bloodstream disorder region IBF is present. The size of the storage space LR may be any size as long as a region having a diameter of several millimeters can be secured depending on the size of the bloodstream disorder region IBF. It goes without saying that it is possible to excise a considerable portion of the vitreous body VB.

After performing the excision of the vitreous body VB (step S100) in this way, the surgeon sets a cannula 90 in the storage space LR and feeds an irrigating solution into the cannula 90 (step S110). A supply tube 92 is connected to the cannula 90. The diluted solution of the oxiglutatione solution is supplied to the cannula 90 via the supply tube 92. In FIG. 7, the most portion of the eyeball is drawn as being filled with the irrigating solution. However, at least the storage space LR only has to be filled with the diluted solution of the oxiglutatione solution. As a result, the diluted solution of the oxiglutatione solution is stored in the upper part of the bloodstream disorder region IBF.

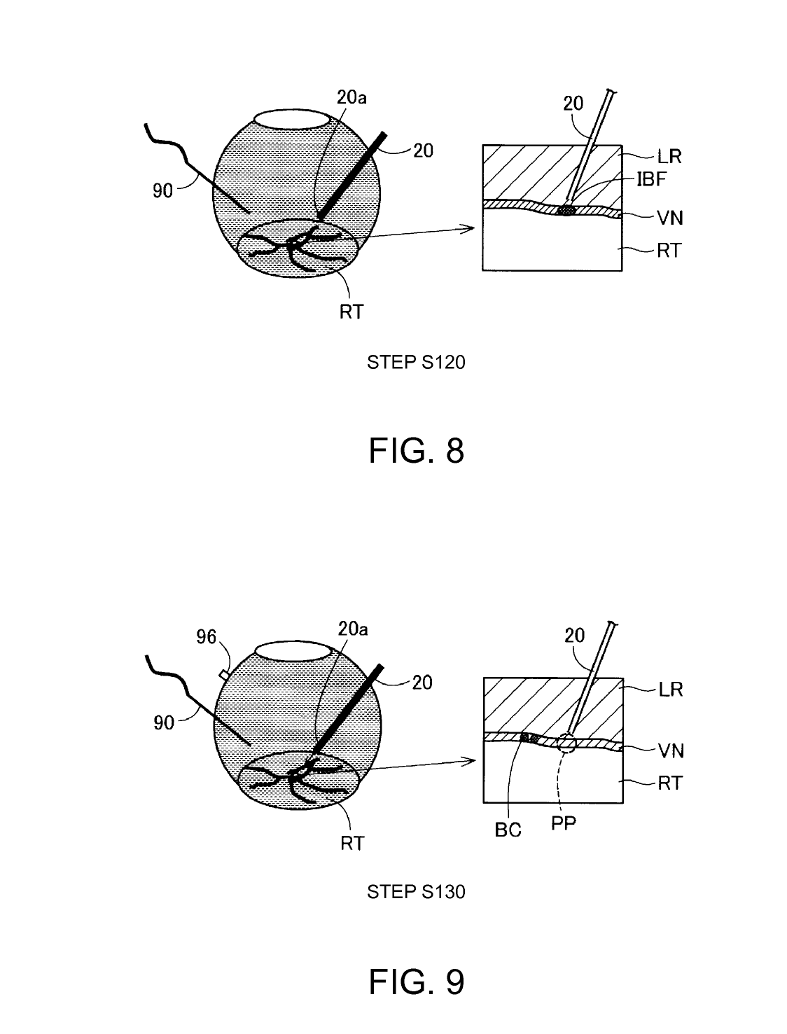

In a state in which the cannula 90 is disposed in the eyeball and the diluted solution of the oxiglutatione solution is irrigated at a predetermined flow rate, the surgeon inserts the liquid ejecting needle 20 of the liquid ejection device 70 into the eyeball and immerses the liquid ejecting needle 20 in the diluted solution of the oxiglutatione solution in the storage space LR (step S120). At this point, the opening 20a of the liquid ejecting needle 20 comes close to the bloodstream disorder region IBF from the upstream side of the bloodstream and is inserted into the vicinity of the bloodstream disorder region IBF, usually, to a position about 0.5 to 2 mm apart from the bloodstream disorder region IBF. This state is shown in FIG. 8. The surgeon inserts the liquid ejecting needle 20 of the liquid ejection device 70 while visually recognizing the liquid ejecting needle 20 using a not-shown microscope inserted into the eyeball or using a magnifying lens through the crystalline lens CL.

After securing a state in which the opening 20a of the liquid ejecting needle 20 is slightly separated from the bloodstream disorder region IBF, the surgeon operates the footswitch 18, drives the liquid ejection device 70, and ejects the liquid from the opening 20a for a predetermined period (step S130). When the footswitch 18 is operated and turned on, the control apparatus 60 outputs a drive signal to the liquid supply mechanism 12 and feeds the diluted solution of the oxiglutatione solution stored in the liquid container 10 into the pulsation generating unit 22. Further, the control apparatus 60 outputs a drive signal to the pulsation generating unit 22 of the hand piece 14 and causes the pulsation generating unit 22 to generate a high-pressure pulsating flow. The liquid (the diluted solution of the oxiglutatione solution) applied with the high pressure is ejected as a pulsating flow from the opening 20a via the liquid ejecting needle 20.

The opening 20a is immersed in the storage space LR in which the diluted solution of the oxiglutatione solution is stored. Therefore, the liquid ejected from the opening 20a is ejected to the liquid stored in advance. The pressure of the liquid spreads in the stored liquid toward the bloodstream disorder region IBF. This state is shown in FIG. 9. While the liquid ejection device 70 is performing intermittent ejection of the liquid, the blood vessel VN on the retina RT is massaged by the liquid around the blood vessel VN. The ejection of the liquid from the opening 20a is performed from the upstream side of the blood vessel VN toward the downstream direction. Therefore, a blood clot BC in the massaged blood vessel VN is washed away to the downstream side. As a result, occlusion in a region where a bloodstream disorder is present in a retinal vein (sign PP in FIG. 9) is eliminated.

When the liquid ejection device 70 operates and the ejection of the liquid from the opening 20a of the liquid ejecting needle 20 continues, a total amount of the liquid in the storage space LR increases. An amount of the liquid is likely to increase according to a perfusion amount by the cannula 90. As a result, if the pressure in the storage space LR is likely to increase, as shown in FIG. 9, a pressure release port 96 only has to be provided. The pressure release port 96 may be a simple opening or may be a dedicated member including a check valve mechanism. It goes without saying that a suction and discharge mechanism may be imparted to the cannula 90 to control the amount of the liquid in the storage space LR.

After confirming that the occlusion of the retinal vein is eliminated, the surgeon performs treatment for completing the surgical operation such as removal of the liquid ejection device 70 and the cannula 90, discharge of the diluted solution of the oxiglutatione solution filled and stored in the storage space LR, filling of an alternative material of the excised vitreous body VB, and suture, sterilization, and cleaning of the surgical part (step S140). The surgeon ends treatment of the retinal vein occlusion.

With the medical treatment method for retinal vein occlusion by the medical treatment system 100 in this embodiment explained above, it is possible to suitably perform the medical treatment of the retinal vein occlusion. The retinal vein is a tissue that is fragile and easily damaged. However, in the medical treatment system 100 in this embodiment, the liquid ejection by the liquid ejection device 70 is intermittently performed and the liquid from the opening is ejected in the liquid stored in the storage space and the pressure of the liquid is reduced. Therefore, the retinal vein is not damaged when the blood vessel is massaged to wash away the blood clot. Moreover, since the pressure of the liquid ejected from the opening is high (in this embodiment, about 0.1 MPa), even in a state in which the opening is separated from the blood vessel, it is possible to sufficiently obtain a massage effect for the blood vessel. It is considered that a surgical instrument should be prevented from directly coming into contact with a fragile tissue such as the retinal vein. Therefore, it is desirable that the surgical operation can be performed with the surgical instrument kept separated from the tissue.

In the medical treatment of the retinal vein occlusion performed using the medical treatment system 100 in this embodiment, the same liquid (the diluted solution of the oxiglutatione solution) is used as the liquid (equivalent to first liquid) stored in the liquid container 10 and ejected from the liquid ejecting needle 20 of the liquid ejection device 70 and the liquid (equivalent to second liquid) stored in the storage space LR formed by removing the vitreous body VB. Therefore, the ejected liquid is mixed with the stored liquid and plays a role of reducing a shock to the tissue on which the liquid is ejected. Since the same liquid is used, there is no concern that the mixed liquid of both the liquids gives some influence on the tissue.

However, the first liquid and the second liquid may be different liquids. For example, in medical treatment, it is sometimes desired to use a drug having some effect for the retina and the blood vessel. When the drug affects the ejecting in the liquid ejection device 70, it is conceivable to add the drug only to the liquid (the second liquid) filled in the storage space to vary the liquids. When it is desired to cause the drug to locally act, if the drug is added to the liquid (the second liquid) filled in the storage space, it is likely that the drug is excessively diluted. In such a case, it is possible to cause the drug to locally act by adding the drug to the liquid (the first liquid) ejected from the liquid ejection device 70. Alternatively, if the stored liquid is made colorless and transparent and the liquid ejected from the liquid ejection device 70 is colored, it is possible to visually understand a state of the ejection in the stored liquid and make use of the state of the ejection for medical treatment.

### C. Second Embodiment

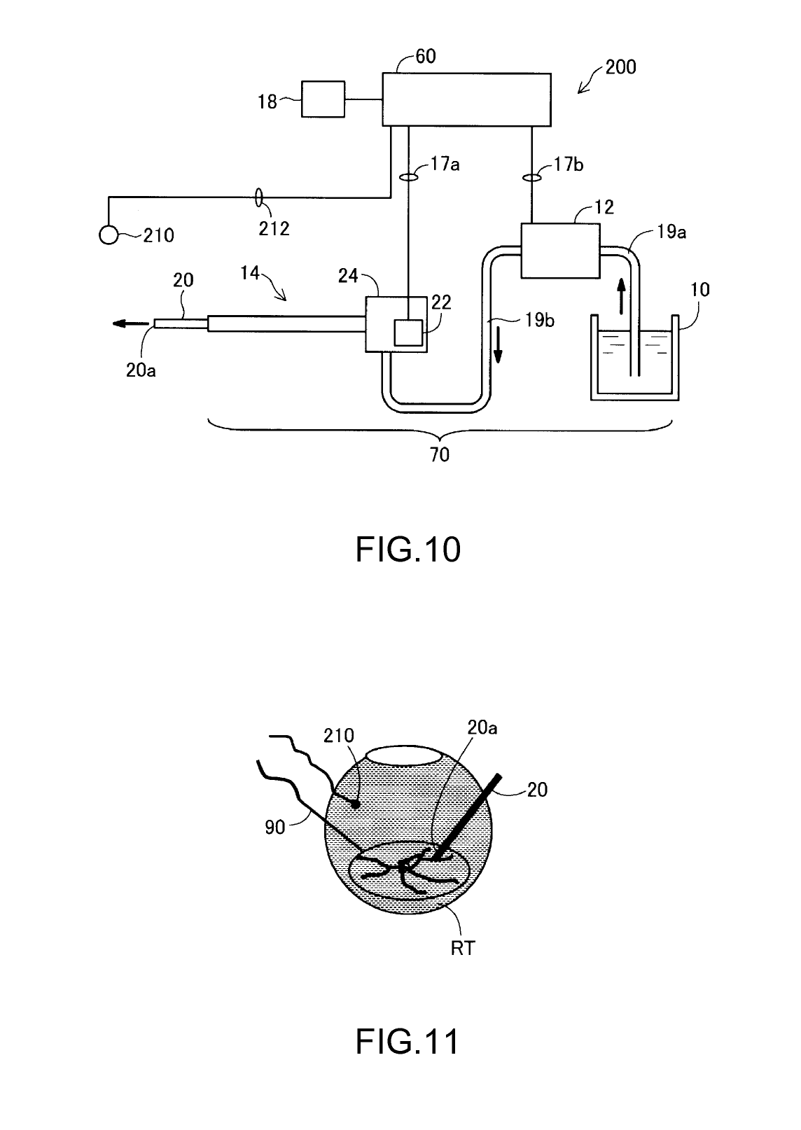

A second embodiment is explained. FIG. 10 is a schematic explanatory diagram showing a medical treatment system according to the second embodiment of the invention. As shown in the figure, in a medical treatment system 200, the liquid ejection device 70 and the control apparatus 60 which are the same as those used in the first embodiment are used. However, the medical treatment system 200 is different from the medical treatment system 100 in the first embodiment in that a pressure sensor 210 is connected to the control apparatus via a connection cable 212. Therefore, explanation concerning the liquid ejection device 70 is omitted. A medical treatment method in the second embodiment is carried out as steps S100 to S140 shown in FIG. 5 as in the first embodiment.

The pressure sensor 210 includes a measurement range and accuracy enough for measuring the pressure in an eyeball. The pressure sensor 210 is disposed in the eyeball and used as illustrated in FIG. 11 in which a state of use is schematically shown. In an example shown in FIG. 11, the pressure sensor 210 is disposed in a formed storage space after at least step S100 is performed among the steps of the medical treatment method in the first embodiment. Detection of pressure by the pressure sensor 210 is performed in step S130.

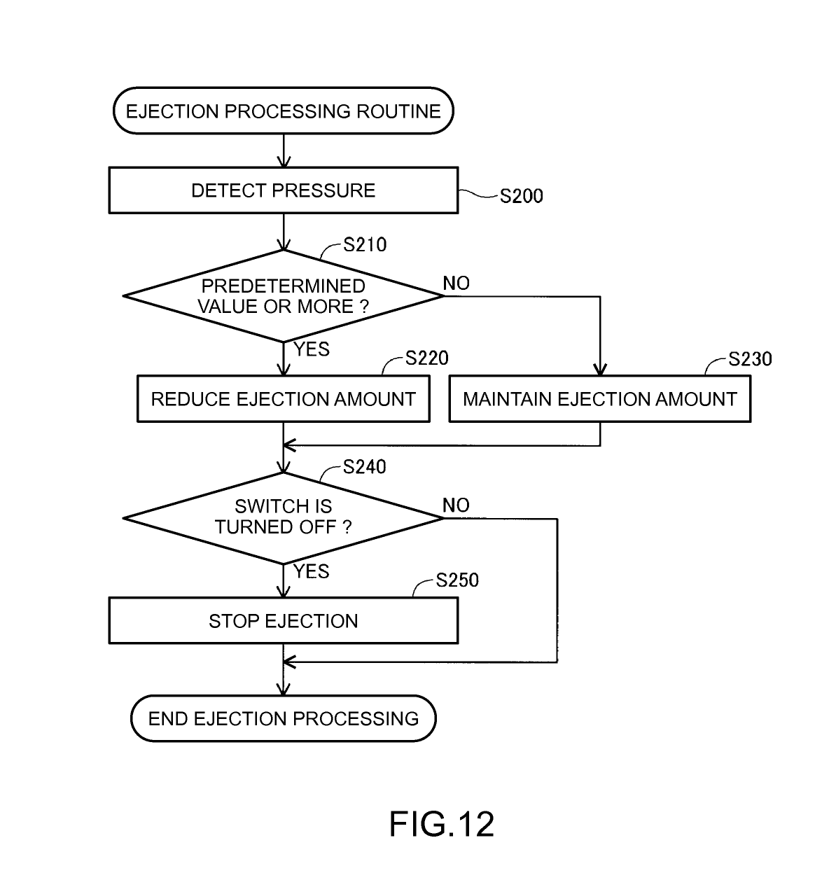

Ejection processing performed by the control apparatus 60 when liquid is ejected from the liquid ejection device 70 for a predetermined period is explained in step S130. FIG. 12 is a flowchart for explaining an ejection control routine executed by the control apparatus 60 in step S130. The control apparatus 60 executes processing shown in FIG. 12 using the microcomputer incorporated therein. The processing shown in FIG. 12 is processing repeatedly executed when the footswitch 18 is operated and turned on. As explained in the first embodiment, when the footswitch 18 is turned on, the control apparatus 60 outputs a drive signal to the pulsation generating unit 22 in the hand piece 14 and starts pulsating flow ejection of the liquid from the opening 20a of the liquid ejecting needle 20. Therefore, the processing shown in FIG. 12 is performed in parallel to such ejection processing.

When the processing shown in FIG. 12 is started, first, the control apparatus 60 reads a signal output from the pressure sensor 210 and detects the pressure of liquid stored in a storage space formed in the eyeball (step S200). Subsequently, the control apparatus 60 determines whether the detected pressure is a predetermined value or more (step S210).

If the pressure of the liquid filled in the storage space is the predetermined value or more (YES in step S210), the control apparatus 60 performs processing for changing the drive signal output to the pulsation generating unit 22 and reducing an ejection amount of the liquid per unit time by a predetermined value (step S220). The reduction of the ejection amount can be easily controlled by reducing the maximum voltage of the drive signal (see FIG. 3) and reducing the duty of the drive signal. Alternatively, the reduction of the ejection amount can also be implemented by changing the drive signal output to the liquid supply mechanism 12 and reducing a supply amount of the liquid by the liquid supply mechanism 12.

On the other hand, if the pressure detected by the pressure sensor 210 is not the predetermined value or more (NO in step S210), the control apparatus 60 maintains the ejection amount (step S230). For convenience of understanding, step S220 of “maintaining the ejection amount” is provided. However, if the change of the ejection amount is not performed, no action is taken, whereby the ejection amount is maintained. After the processing in steps S220 and S230, the control apparatus 60 determines whether the footswitch 18 is turned off (step S240). If the footswitch 18 is turned off, the control apparatus 60 stops the output of the drive signal to the liquid supply mechanism 12 and the pulsation generating unit 22 and stops the ejection (step S250). If the footswitch 18 is not turned off, the control apparatus 60 ends this processing routine once without taking any action. Note that, instead of the stop of the ejection, warning indicating a pressure rise may be performed by sound, lighting of a lamp, or the like to warn a surgeon about the pressure rise.

According to the second embodiment explained above, the medical treatment which is the same as the medical treatment in the first embodiment can be implemented. Further, the ejection amount is reduced when the liquid in the storage space increases as a result of ejecting the liquid from the liquid ejection device 70 and the pressure of the liquid in the storage space rises. Therefore, it does not occur that the pressure in the storage space uselessly rises to damage a tissue such as a retina. Even when an amount of the liquid supplied by the ejection is large and a leak of the liquid from the pressure release port 96 illustrated in FIG. 9 is insufficient, if the medical treatment system in the second embodiment is used, it is possible to perform control to prevent the pressure in the storage space from exceeding the predetermined value.

### D. Modifications

### Modification 1

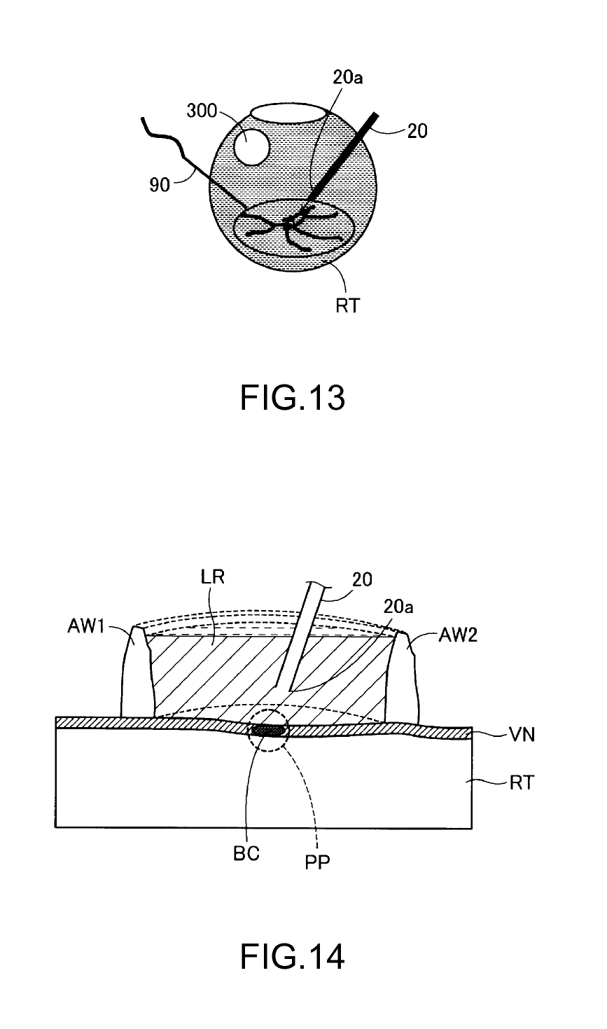

In the embodiments, the pressure of the liquid in the storage space is prevented from excessively rising by providing the pressure release port 96 and by performing the control of the ejection amount based on the pressure detected by the pressure sensor 210. However, other methods may be used. For example, as illustrated in FIG. 13, pressure fluctuation in the storage space may be absorbed by providing, in the storage space, an accumulator 300 that absorbs the pressure fluctuation. As the accumulator 300, a simple component such as a bag of an elastic body or a non-elastic body, in which gas such as the air is encapsulated, can also be used.

### Modification 2

In the first and second embodiments, since the medical treatment target is the retinal vein, the storage space is formed in the eyeball and the liquid is ejected from the opening 20a of the liquid ejecting needle 20 in the liquid filled in the storage space. On the other hand, depending on a medical treatment region, as shown in FIG. 14, a wall AW may be provided around a part PP, which is a medical treatment target of the blood vessel VN. The liquid may be filled in the space LR formed by the wall AW surrounding the medical treatment part PP. The liquid ejecting needle 20 of the liquid ejection device 70 may be inserted into the space LR to perform ejection of the liquid. Consequently, as in the first and second embodiments, it is possible to massage the blood vessel VN, wash away the blood clot BC or the like on the inside of the blood vessel VN downstream, and eliminate occlusion of the blood vessel VN without applying a heavy shock to the blood vessel VN.

FIG. 14 is an explanatory diagram showing a part of the wall as end faces. Two end faces AW1 and AW2 of the wall are drawn. The wall is formed to surround the medical treatment part PP. As the wall, a biocompatible material (e.g., silicon) or the like may be formed in a ring shape in advance and simply placed on a tissue RT or may be bonded to the tissue RT by a biodegradable adhesive. Alternatively, by irradiating light for curing while pressing out a photocurable material from a distal end of a nozzle, prior to medical treatment, a wall may be formed in the place. If the liquid only has to be stored for a short time, the liquid may be stored in a state of a gel-like wall without curing the material. The gel-like material can also be sucked by a suction tube or the like and removed after a surgical operation.

### Modification 3

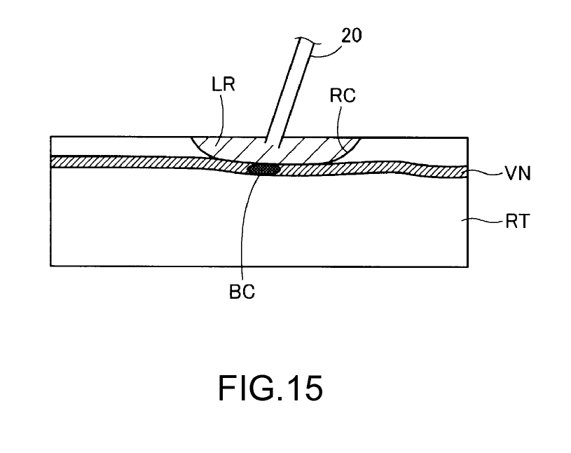

When the blood vessel VN is present under the tissue RT having predetermined thickness, as shown in FIG. 15, the tissue may be excised to form a hollow RC. The liquid may be stored in a hollowed space LR. If the liquid ejecting needle 20 of the liquid ejection device 70 is inserted into the stored liquid and the liquid is ejected from the distal end of the liquid ejecting needle 20, as in the first and second embodiments, it is possible to massage the blood vessel VN, wash away the blood clot BC or the like on the inside of the blood vessel VN downstream, and eliminate occlusion of the blood vessel VN without applying a heavy shock to the blood vessel VN.

### Modification 4

In the embodiments, the pulsation generating unit 22 for ejecting a pulsating flow is configured using the piezoelectric element. However, other mechanisms may be used as long as large pressure fluctuation can be generated in a short time. For example, a bubble generating unit may be provided in the space in which the first liquid is stored. Pulsation may be generated using a pressure rise involved in occurrence of bubbles due to boiling of the liquid. For the boiling of the liquid in the bubble generating unit, heating means such as a resistor heater, a ceramic heater, a microwave, and an optical maser can be used.

### Other Modifications

The invention can be carried out in other various forms. For example, in the embodiments, the invention is used for the medical treatment of the retinal vein occlusion. However, the invention can be widely applied to occlusion other than the retinal vein occlusion. For example, the invention may be applied to relaxation of pulmonary thromboembolism and occlusion of a blood vessel in a brain such as thrombotic brain infarction. The invention may be applied to occlusion of, for example, a coronary artery of a heart. The invention may be used for, for example, medical treatment of thromboangiitis obliterans and arteriosclerosis obliterans. Alternatively, the invention can also be applied to, for example, medical treatment of deep vein thrombosis (so-called economy class syndromes) and medical treatment of primary varix. A medical treatment target may be animals in general including human or may be limited to animals excluding human.

The method of the invention is a method of ejecting pulsating liquid into liquid to massage a blood vessel. Therefore, an excellent effect is obtained when the method is applied to an extremely fragile blood vessel such as a retinal vein. Therefore, the effect is large when the method is applied to medical treatment of occlusion of a blood vessel made brittle because of worsened sclerosis, a capillary, and the like. It goes without saying that the method may be used for medical treatment of occlusion of a normal blood vessel. Even in this case, an excellent medical treatment effect is also obtained in that damage to the blood vessel is small.

In the medical treatment system in the invention, the insertion section is inserted into a tissue of an animal and used. In the first and second embodiments, it is explicitly understood that the opening 20a, which is the distal end of the insertion section, is inserted into the tissue in the eyeball. On the other hand, in the modification 2 and the modification 3, the space LR is opened. However, even in this case, since the blood vessel VN is present in the body, it is understood that the opening 20a, which is the distal end of the insertion section, is inserted into a tissue at a point in time when the opening 20a crosses the surface (the boundary) of a skin or an organ (not shown in the figure).

The invention is not limited to the embodiments and the modifications explained above and can be implemented as various configurations without departing from the spirit of the invention. For example, the technical features in the embodiments, the examples, and the modifications corresponding to the technical features in the aspects described in the summary of the invention can be replaced or combined as appropriate in order to solve a part or all of the problems or attain a part or all of the effects. Unless the technical features are explained in this specification as essential technical features, the technical features can be deleted as appropriate.

What is claimed is:

1. A medical treatment method for a bloodstream disorder, comprising:
preparing a ejection device that includes an insertion section insertable into a tissue of an animal and is capable of ejecting first liquid as a pulsating flow from a distal end of the insertion section; and
bringing the distal end of the insertion section close to a blood vessel in the tissue, which is a medical treatment target, and ejecting the first liquid as the pulsating flow from the distal end of the insertion section in a state in which second liquid is interposed in a region covering the blood vessel.

2. The medical treatment method according to claim 1, wherein the first liquid and the second liquid are same liquid.

3. The medical treatment method according to claim 1, wherein prior to the ejection of the first liquid, the second liquid is filled in the region covering the blood vessel.

4. The medical treatment method according to claim 3, wherein, prior to the filling of the second liquid, at least a part of the tissue present in the region covering the blood vessel is excised to form a space in which the second liquid is filled.

5. The medical treatment method according to claim 1, wherein, when the ejection of the first liquid is performed, an increase in pressure in the region where the second liquid is interposed is reduced.

6. The medical treatment method according to claim 5, wherein the reduction in the pressure rise is performed by discharge of the second liquid from the region.

7. The medical treatment method according to claim 5, wherein the reduction in the pressure rise is performed by increasing or reducing, according to the pressure in the region, a volume of a pressure reduction chamber provided in the region.

8. The medical treatment method according to claim 1, wherein pressure in the region is monitored to carry out at least one of adjustment of the pressure based on fluctuation in the pressure during medical treatment, a stop of the ejection of the first liquid, and provision of the pressure fluctuation during the medical treatment to a surgeon.

9. The medical treatment method according to claim 1, wherein the tissue of the animal is an eyeball, the blood vessel is a retinal vein of the eyeball, and the bloodstream disorder is retinal vein occlusion.

10. A medical treatment apparatus used for the medical treatment method according to claim 1, the medical treatment apparatus comprising a ejection device that includes an insertion section insertable into a tissue of an animal and is capable of ejecting first liquid as a pulsating flow from a distal end of the insertion section.

11. A medical treatment system for a bloodstream disorder, comprising:
a ejection device that includes an insertion section insertable into a tissue of an animal and is capable of ejecting first liquid as a pulsating flow from a distal end of the insertion section; and
a control apparatus configured to bring the distal end of the insertion section close to a blood vessel in the tissue, which is a medical treatment target, and eject the first liquid as the pulsating flow from the distal end of the insertion section in a state in which second liquid is interposed in a region covering the blood vessel.

12. The medical treatment system according to claim 11, further comprising an excising apparatus configured to excise the tissue present in the region covering the blood vessel and form a region where the second liquid is stored.

13. The medical treatment system according to claim 12, further comprising a liquid supplying apparatus configured to fill the second liquid in the formed region.

14. The medical treatment system according to claim 11, further comprising a pressure detecting apparatus configured to detect pressure of the second liquid interposed in the region covering the blood vessel.

15. The medical treatment system according to claim 14, wherein the control apparatus controls an ejection amount of the first liquid from the ejection device using the detected pressure.

16. A medical treatment apparatus used for the medical treatment method according to claim 2, the medical treatment apparatus comprising a ejection device that includes an insertion section insertable into a tissue of an animal and is capable of ejecting first liquid as a pulsating flow from a distal end of the insertion section.

17. A medical treatment apparatus used for the medical treatment method according to claim 3, the medical treatment apparatus comprising a ejection device that includes an insertion section insertable into a tissue of an animal and is capable of ejecting first liquid as a pulsating flow from a distal end of the insertion section.

18. A medical treatment apparatus used for the medical treatment method according to claim 4, the medical treatment apparatus comprising a ejection device that includes an insertion section insertable into a tissue of an animal and is capable of ejecting first liquid as a pulsating flow from a distal end of the insertion section.

19. A medical treatment apparatus used for the medical treatment method according to claim 5, the medical treatment apparatus comprising a ejection device that includes an insertion section insertable into a tissue of an animal and is capable of ejecting first liquid as a pulsating flow from a distal end of the insertion section.

20. A medical treatment apparatus used for the medical treatment method according to claim 6, the medical treatment apparatus comprising a ejection device that includes an insertion section insertable into a tissue of an animal and is capable of ejecting first liquid as a pulsating flow from a distal end of the insertion section.
